(19) United States
(12) Patent Application Publication
(10) Pub. No.: US 2025/0041515 A1
(43) Pub. Date: Feb. 6, 2025

(54) System and Method for Imaging Implanted Medical Devices

(71) Applicant: Bard Access Systems, Inc., Salt Lake City, UT (US)

(72) Inventors: Shayne Messerly, Kaysville, UT (US); Manish Kumar, Bengaluru (IN); Praveen Nalawade, Bengaluru (IN); Karthik Mysuru Ramesh Kumar, Bangalore (IN); Shishir Prasad, Ramsey, NJ (US); Jonathan Karl Burkholz, Salt Lake City, UT (US)

(21) Appl. No.: 18/789,564

(22) Filed: Jul. 30, 2024

Related U.S. Application Data
(60) Provisional application No. 63/530,003, filed on Jul. 31, 2023.

(51) Int. Cl. A61M 5/158

(52) U.S. Cl. CPC A61M 5/158; A61M 2005/1586; A61M 2005/1588; A61M 2205/0238; A61M 2205/3306

(57) ABSTRACT

A vascular access device (VAD) monitoring system includes a VAD with a catheter tube that is subcutaneously disposed and incorporates a fluorescent dye. An optical imaging system is also provided, comprising a light source emitting infrared (IR) excitation light and a camera capable of detecting signal light emitted by the fluorescent dye upon exposure to the excitation light. This enables real-time monitoring of the VAD, allowing for the detection of any potential complications or issues related to the vascular access device. The combination of the catheter tube with the fluorescent dye and the optical imaging system provides a non-invasive and efficient means of monitoring the VAD, enhancing patient safety and improving overall healthcare outcomes.

### PRIORITY

This application claims the benefit of priority to U.S. Provisional Application No. 63/530,003, filed Jul. 31, 2023, which is incorporated in its entirety into this application.

### BACKGROUND

Briefly summarized, embodiments of the present invention are directed to systems and methods for the placing and monitoring of sub-dermal implanted medical devices by optical imaging. Various complications can occur during the placement and/or during the useful life span, or dwell time, of sub-dermal implanted medical devices, such as catheters and/or ports. Exemplary complications incurred include misplacement of the distal tip, missing the target vessel, accessing the incorrect vessel, backwalling, distal tip placement adjacent a valve or bifurcation, infiltration, extravasation, dislodgement, occlusion, loss of patency, infection, catheter collapse, catheter kinking, catheter movement, thrombus development, phlebitis, or the like. Further localized changes in a patient's physiology can further affect the functional dwell time of the medical device, for example vein or arterial size changes, collapse, stiffening, damage, or the like may lead to further complications. Such complications can be difficult to diagnose without disturbing the placement of the medical device. Often such devices are removed prematurely due to a false positive diagnosis of a problem. Alternatively, such complications can go undetected leading to reduced efficacy of the treatment or increased patient morbidity.

Further, complications with the placement of the medical device can be equally challenging. Choosing the placement location, placing the medical device, and confirming correct placement of the medical device often cannot be directly observed and as such clinicians either rely on fluoroscopic imaging to confirm correct placement or rely on secondary indicators to identify any problems in the placement procedure. Fluoroscopic imaging exposes the patient to harmful radiation and relying on secondary indicators fails to preempt problems that may otherwise be avoidable if they had been detected earlier. Optionally, clinicians utilize ultrasonic imaging techniques to image sub-dermal VAD's. However, these imaging techniques suffer from a limited field of view and are susceptible to reflectivity problems that introduce “noise” to the image and/or fail to distinguish between tissue structures or medical device structures that have similar acoustic impedance properties.

What is needed therefore is a system and method to assess and monitor the position, status and viability of indwelling VAD's without relying on potentially harmful imaging techniques. Such systems can confirm correct placement and compare the current status of an indwelling VAD with previous states, or compared with established standards of care, to predict and identify VAD related complications. Such systems can be important in the acute care and alternate care settings to reduce the patient complications and experience, reduce clinician burden, and improve overall effectiveness of the patient's treatment and care.

### SUMMARY

In some aspects, the techniques described herein relate to a vascular access device (VAD) monitoring system including, a VAD having a catheter tube disposed distally and configured to be disposed subcutaneously, the catheter tube including a dye, and an optical imaging system including, a light source configured to emit excitation light, and a camera configured to detect signal light emitted from the dye when the dye is exposed to the excitation light.

In some aspects, the techniques described herein relate to a VAD monitoring system wherein the excitation light includes electromagnetic radiation in a range of 700 nm-1 mm.

In some aspects, the techniques described herein relate to a VAD monitoring system wherein the excitation light includes electromagnetic radiation in a range of 700 nm-2500 nm.

In some aspects, the techniques described herein relate to a VAD monitoring system wherein the dye is formed integrally with a wall of the catheter tube.

In some aspects, the techniques described herein relate to a VAD monitoring system, wherein the dye is included in a coating disposed on a surface of the catheter tube.

In some aspects, the techniques described herein relate to a VAD monitoring system wherein the excitation light has a first wavelength range and the signal light has a second wavelength range, different from the first wavelength range.

In some aspects, the techniques described herein relate to a VAD monitoring system wherein the VAD further includes a dressing configured to adhere to a skin surface of a patient and includes a fiduciary marker configured to align one or both of the light source and the camera with a portion of the catheter tube disposed subcutaneously therebelow.

In some aspects, the techniques described herein relate to a VAD monitoring system wherein the light source and the camera are provided as a single handheld device.

In some aspects, the techniques described herein relate to a VAD monitoring system wherein the light source and the camera are provided as separate stand-alone devices.

In some aspects, the techniques described herein relate to a VAD monitoring system wherein the light source is included on the dressing.

In some aspects, the techniques described herein relate to a VAD monitoring system wherein the optical imaging system includes an excitation light logic, a signal light logic, and an image analysis logic configured to analyze the signal light detected by the camera and detect a change in the signal light relative to a threshold image.

In some aspects, the techniques described herein relate to a VAD monitoring system wherein the threshold image generated from one or more previous images of the VAD is generated by the optical imaging system.

In some aspects, the techniques described herein relate to a VAD monitoring system wherein the optical imaging system determines a quantified change in the signal light and displays the quantified change as a metric on a display of the optical imaging system.

In some aspects, the techniques described herein relate to a VAD monitoring system wherein the image analysis logic of the optical imaging system is configured to analyze the signal light and generate an image of the catheter tube and determine one or more of an occlusion, a thrombosis, an abluminal biofilm, an intraluminal biofilm, a fibrin sheath, a catheter tube dislodgement, a loss of patency of the catheter tube, a catheter tube collapse, damage to the catheter tube, a proximity of a distal tip of the catheter tube to a vascular valve, or a proximity of the distal tip of the catheter tube to a vascular bifurcation.

In some aspects, the techniques described herein relate to a method of detecting a complication with a vascular access device (VAD) disposed within a patient including, providing excitation light to a skin surface of the patient, impinging the excitation light on a dye included with a catheter tube of the VAD disposed subcutaneously, emitting signal light from the dye, detecting the signal light by a camera disposed externally to the patient, determining an image of the VAD, and analyzing the image to determine if the complication is present based on a change in the signal light relative to a threshold image data.

In some aspects, the techniques described herein relate to a method, further including providing the excitation light in an infrared (IR) or near infrared (NIR) spectra.

In some aspects, the techniques described herein relate to a method wherein the dye is formed integrally within a wall of the catheter tube.

In some aspects, the techniques described herein relate to a method wherein the dye is included in a coating disposed on a surface of the catheter tube.

In some aspects, the techniques described herein relate to a method wherein the threshold image data is generated from one or more previous images of the VAD.

In some aspects, the techniques described herein relate to a method, wherein the complication includes one or more of an occlusion, a thrombosis, an abluminal biofilm, an intraluminal biofilm, a fibrin sheath, a catheter tube dislodgement, a loss of patency of the catheter tube, a catheter tube collapse, damage to the catheter tube, a proximity of a distal tip of the catheter tube to a vascular valve, and a proximity of the distal tip of the catheter tube to a vascular bifurcation.

In some aspects, the techniques described herein relate to a medical device imaging system including, a medical device having a catheter tube and including a distal portion disposed intravascularly, the catheter tube including a dye, and an optical imaging system disposed externally and including a light source, a camera, and a console including one or more logic modules stored within a non-transitory storage medium, the one or more logic modules, when executed by one or more processors, perform operations including, emitting excitation light from the light source to impinge on the dye included with the distal portion of the catheter tube disposed intravascularly, detecting signal light emitted from the dye of the distal portion to provide an image, parsing the image to determine a shape of the distal portion of the medical device, and analyzing the parsed image against threshold data to determine a presence of a structural abnormality or a deposit.

In some aspects, the techniques described herein relate to a medical device imaging system, wherein the excitation light includes electromagnetic radiation in an infrared or a near infrared range.

In some aspects, the techniques described herein relate to a medical device imaging system wherein the dye is formed integrally with a wall of the catheter tube or is included in a coating disposed on a surface of the catheter tube.

In some aspects, the techniques described herein relate to a medical device imaging system wherein the excitation light has a first wavelength range and the signal light has a second wavelength range, different from the first wavelength range.

In some aspects, the techniques described herein relate to a medical device imaging system wherein the medical device further includes a dressing configured to adhere to a skin surface of a patient and includes a fiduciary marker configured to align one or both of the light source and the camera with the distal portion of the catheter tube disposed subcutaneously therebelow.

In some aspects, the techniques described herein relate to a medical device imaging system wherein analyzing the parsed image against the threshold data further includes training a machine learning model with a plurality of labeled images, the trained machine learning model configured to determine a structural anomaly or a deposit disposed on the medical device and provide an alert to a user.

In some aspects, the techniques described herein relate to a medical device imaging system wherein the plurality of labeled images include labeled images of a medical device including a deposit, labeled images of a medical device including a structural anomaly, and labeled images of a medical device without either of a deposit or a structural anomaly.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a vascular access device (VAD) monitoring system, in accordance with embodiments disclosed herein.

FIG. 1B shows a perspective view of a first VAD of FIG. 1A coupled with a second VAD, in accordance with embodiments disclosed herein.

FIG. 1C shows close up detail of a distal tip section of the device of FIG. 1B, in accordance with embodiments disclosed herein.

FIG. 2A shows a perspective cutaway view of a distal tip of a VAD disposed within a vessel adjacent a valve, in accordance with embodiments disclosed herein.

FIG. 2B shows a perspective cutaway view of a distal tip of a VAD disposed within a vessel adjacent a bifurcation, in accordance with embodiments disclosed herein.

FIG. 2C shows a schematic side view of a distal portion of a catheter tube disposed within a vessel, in accordance with embodiments disclosed herein.

FIG. 2D shows a schematic plan view of the distal portion of the catheter tube of FIG. 2C disposed sub-dermally and being imaged by the optical imaging system, in accordance with embodiments disclosed herein.

FIG. 3A shows a schematic side view of a portion of the catheter tube disposed sub-dermally and including one or more deposits, in accordance with embodiments disclosed herein.

FIG. 3B shows a schematic plan view the catheter tube of FIG. 3A imaged by an optical imaging system, in accordance with embodiments disclosed herein.

FIG. 4A shows a schematic plan view a catheter tube imaged by an optical imaging system without any deposits or structural deformations, in accordance with embodiments disclosed herein.

FIGS. 4B-4E show schematic plan views of the catheter tube of the FIG. 4A imaged by an optical imaging system and including one or more complications, in accordance with embodiments disclosed herein.

FIG. 5 shows a schematic view of the optical imaging system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

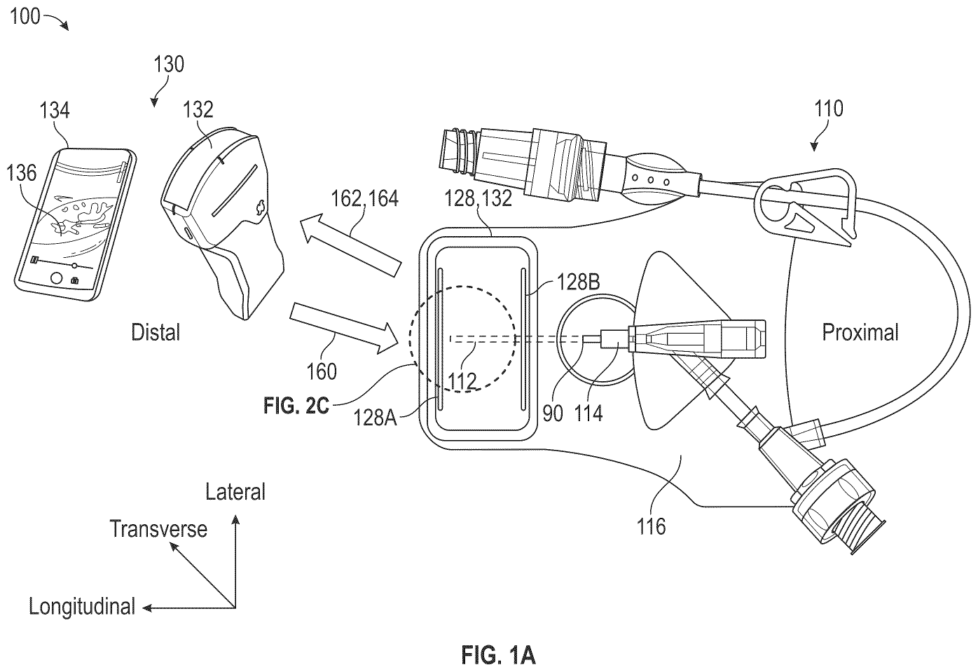

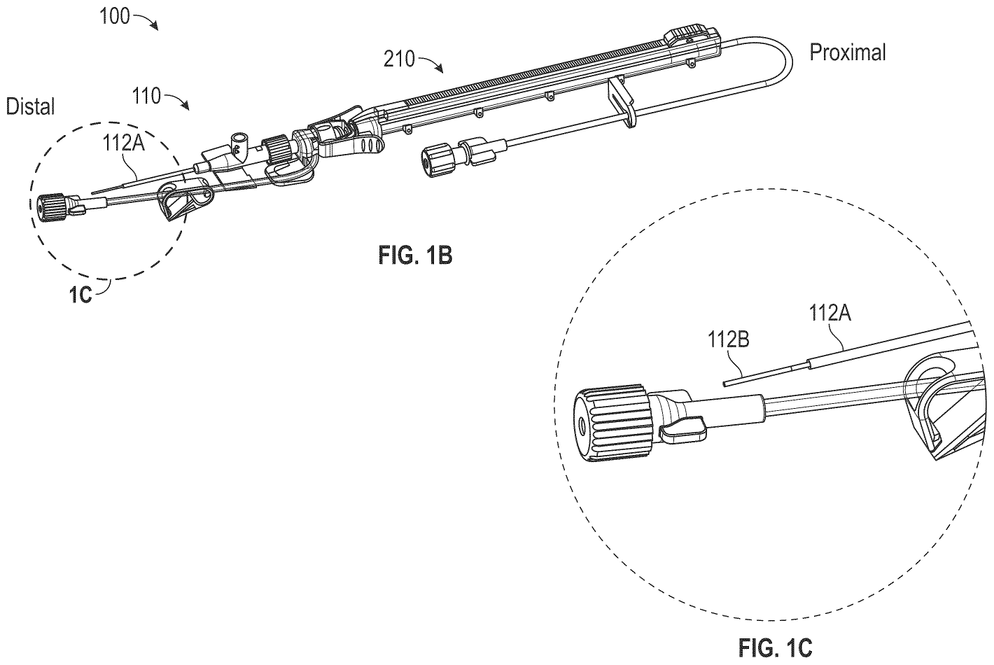

To assist in the description of embodiments described herein, as shown in FIG. 1A, a longitudinal axis extends substantially parallel to an axial length of the catheter tube 112. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1A shows an exemplary vascular access device (“VAD”) monitoring system (“system”) 100 generally including a vascular access device (“VAD”) system 110 and an optical imaging system 130. Exemplary vascular access devices (VAD) 110 can include, but not limited to, single lumen catheters, multi-lumen catheters, midline catheters, intravenous catheters, peripheral intravenous catheters (PIVC), peripheral intravenous outflow devices (PIVO), central venous catheters (CVC), peripherally inserted central catheters (PICC), rapidly insertable central catheters (RICC), access sites, ports, subcutaneous access ports, and the like.

In an embodiment, the VAD 110 includes a catheter tube 112 defining a lumen 118 and extending along a longitudinal axis. The catheter tube 112 is supported at a proximal end by a catheter hub 114. The catheter tube 112 can be formed of a plastic, polymer, elastomer, silicone rubber, rubber, polymethyl methacrylate (PMMA), or similar suitable material. The distal tip of the catheter tube 112 extends percutaneously at an insertion site 90 into the body of a patient to a target location. The catheter hub 114 is configured to remain outside of the body of the patient. It will be appreciated, however, that this is not intended to be limiting and various portions of the VAD 110, or in some embodiments, the entire VAD 110 can be disposed subcutaneously. For example, where the VAD 110 includes a sub-dermal access port coupled to the catheter, the entire VAD 110 can be disposed sub-dermally. In an embodiment, the VAD 110 can further include various numbers or combinations of catheter tubes, hubs, connectors, stabilization wings, extension legs, secondary VAD's, or the like, coupled thereto.

In an embodiment, the VAD system 110 can further include a dressing 116 configured to adhere to a skin surface of the patient and, optionally, a portion of the VAD 110 as well. The dressing 116 can form a barrier over the insertion site 90, mitigating infection. Further, the dressing 116 can stabilize portions of the VAD 110 disposed outside of the body of the patient.

In an embodiment, as shown in FIGS. 1B-1C, the system 100 can include a second VAD 210 coupled with the first VAD 110. For example, the first VAD 110 can be a PIV catheter including a first catheter tube 112A and providing access to a vessel. The second VAD 210 can include a PIVO configured to couple with the hub 114 of the first VAD 110 and slide a second catheter tube 112B through the lumen 118 of the first catheter tube 112A to access the vasculature. In an embodiment, the second VAD 210 can be configured to engage the first VAD 110 to access the vasculature and aspirate a blood sample without having to puncture the vessel at a separate location from the first VAD 110.

In an embodiment, one or more portions of the VAD 110 can include a dye 120 configured to reflect, absorb and/or fluoresce when exposed to electromagnetic (EM) radiation, such as Infrared (IR) or Near Infrared (NIR) spectra. However, it will be appreciated that embodiments described herein can also utilize other EM spectra having greater or lesser wavelengths, e.g., optical or ultraviolet. As used herein the Infrared (IR) spectrum includes wavelengths in a range of between 700 nm and 1 mm. As used herein the Near Infrared (NIR) spectrum includes wavelengths in a range of between 700 nm and 2500 nm.

In an embodiment, the dye 120 is formed integrally with one or more portions of the VAD 110, e.g., formed integrally with a polymer used to form the walls of the catheter tube 112, the hub 114, or the like. In an embodiment, the dye 120 is included in a coating disposed on a surface of the VAD 110, e.g., on an inner surface of the catheter lumen 118 and/or on an outer surface of the catheter tube 112. In an embodiment, the dye 120 is an agglomerated nanoparticle having a primary particle size of between 10-50 nm, however, other primary particle sizes that are greater or smaller are also contemplated. In an embodiment, the dye 120 includes cyanine dyes, antimony tin oxides (ATO), indium tin oxides (ITO), doped tungsten oxides (CTO), combinations thereof, or the like. As will be appreciated, these dyes are exemplary and other dyes are also contemplated.

In an embodiment, the dye 120 is configured to reflect, transmit, and/or absorb EM radiation wavelengths differently from that of the surrounding tissues or fluids. For example, the dye 120 can provide greater reflection, less transmission, and/or less absorption to the EM radiation than blood and less reflection, greater transmission, and/or greater absorption to the EM radiation than the vessel wall tissue. As such, when visualized by the optical imaging system 130, the portion of the VAD 110 including the dye 120 appears more opaque than the blood, but less opaque than vessel wall tissue. As will be appreciated these are exemplary, and other combinations of greater or lesser reflection, transmission, or absorption between the dye 120 and the blood, surrounding tissues, etc. are also contemplated.

In an embodiment, the dye 120 can be configured to fluoresce under EM radiation. For example, the dye 120 can be configured to absorb EM radiation of a first wavelength and, in response, emit EM radiation of a second wavelength, different from the first wavelength. The second wavelength can be configured to be different from the reflected wavelength(s) from the surrounding tissues/blood in order to differentiate the VAD 110, or portions thereof, from these surrounding tissues/fluid, as described herein.

In an embodiment, different concentrations of dye 120 can affect the amount of reflection, transmission, or absorption of the EM radiation through the VAD 110. For example, relatively higher concentrations of dye 120 will provide relatively higher reflection of EM radiation and provide relatively greater reflection or absorption of EM radiation. Different colors of dye 120 can provide different absorption of EM radiation. For example, black or dark colors provide greater absorption of EM radiation where white or light colors provide greater reflection of EM radiation. Different surface characteristics of the dye particles 120 provide different reflective properties of EM radiation. For example, smoother surfaces of the dye particles provide greater reflection of EM radiation relative to rougher, or more uneven surfaces of dye particles. Lastly, different fluorescent properties of the dye 120 can provide different emitted light properties. For example, the dye 120 can absorb EM radiation of a first wavelength, or range of wave lengths, and emit EM radiation of a second wavelength, or range of wavelengths, as described herein. Accordingly, modifying theses different properties of the dye 120 can facilitate differentiating the dye 120, and associated structures of the VAD 110, from surrounding tissues and fluid when imaged by the optical imaging system 130, as described herein.

With continued reference to FIG. 1A, in an embodiment, the system 100 further includes an optical imaging system 130. The optical imaging system 130 is configured to emit excitation light 160, e.g., from a light source 132, that is capable of penetrating the surface layers of skin 70, sub-dermal tissues 72, and vessel walls 74 to impinge on at least a portion of the VAD 110. Worded differently, the optical imaging system 130 is configured to emit electromagnetic radiation in the Infrared (IR) and/or Near Infrared (NIR) spectra to penetrate the surface layers of skin 70, sub-dermal tissues 72, and vessel walls 74 to impinge on the sub-dermal portions of the VAD 110. In an embodiment, the excitation light 160 would be capable of penetrating the skin surface tissues to a depth of about 2 cm. However, greater or lesser depths are also contemplated.

In an embodiment, the excitation light 160 from a light source 132 impinges on the VAD 110, the dye 120, or combinations thereof, causing the dye 120 to reflect, absorb, and/or fluoresce. The wavelengths of the reflected light 164 can be the same or different from the wavelengths of the excitation light 160. Where the dye 120 is configured to fluoresce, the dye 120 absorbs the excitation light 160 and emits a signal light 162. The signal light 162 being the same or different from the excitation light 160. The optical imaging system 130 further includes a camera 134, or similar device, configured to detect the reflected light 164 and/or signal light 162.

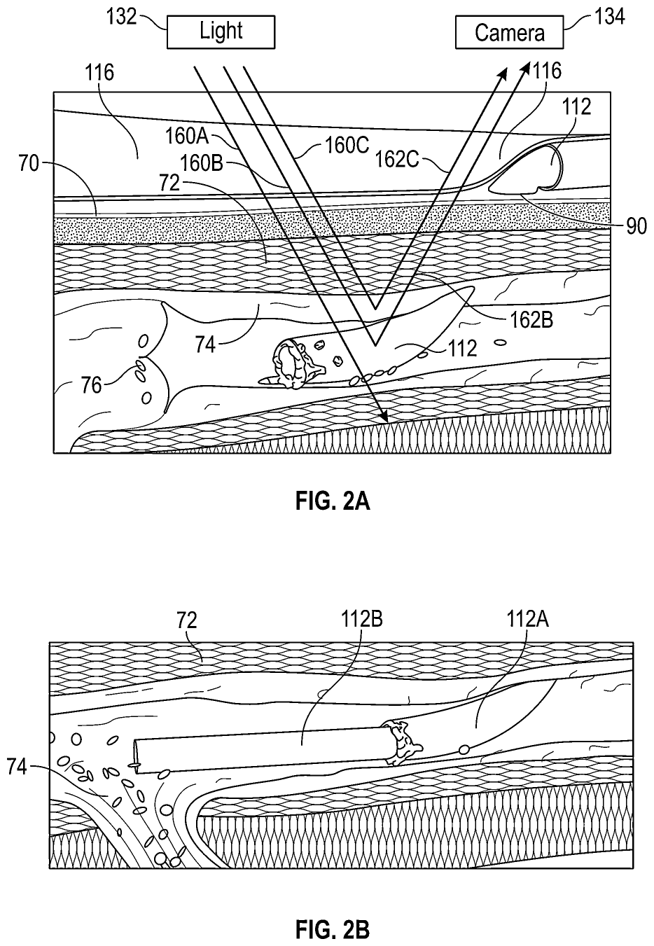

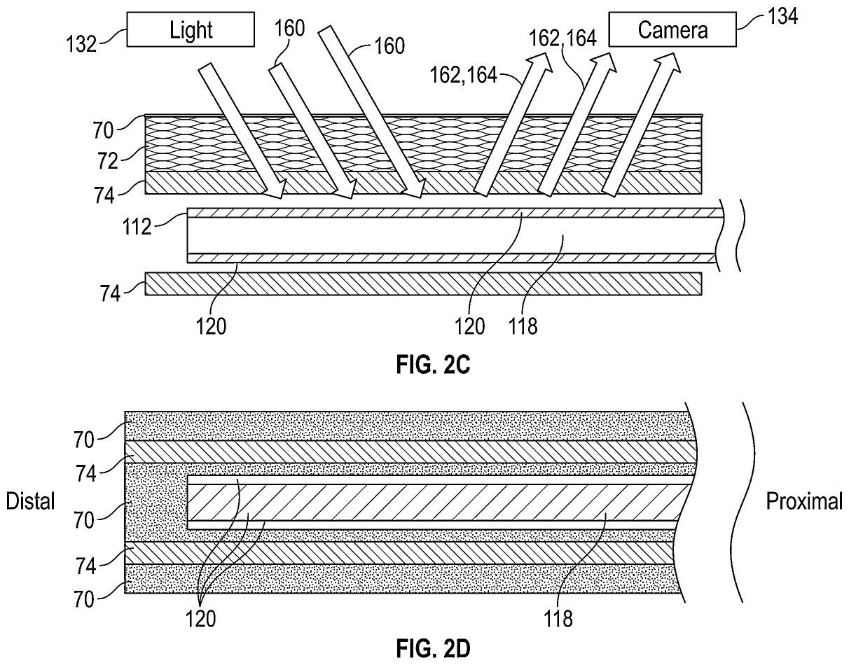

FIGS. 2A-2D show schematic views of a VAD 110 disposed within a vessel 74. FIG. 2A shows a perspective cut-away view of a distal portion of the VAD 110 extending through an insertion site 90 on a skin surface 70 to access a vessel 74. FIG. 2B shows a perspective cut-away view of a distal portion of the VAD 110 having a first catheter tube 112A, and a second catheter tube 112B disposed within the lumen of the first catheter tube 112A to access a vessel 74. FIG. 2C shows a schematic side view of a portion of the catheter tube 112 disposed sub-dermally within a vessel 74. FIG. 2D shows a schematic plan view of a portion of the catheter tube 112 disposed sub-dermally and being imaged by the optical imaging system 130.

The dye 120 disposed in the VAD 110, or in a coating disposed on the VAD 110, is configured to reflect, transmit, absorb, and/or fluoresce to differentiate the VAD 110 from surrounding tissues and fluid. As shown in FIG. 2A, a first excitation light 160A from the light source 132 can transmit through or be absorbed by certain tissues, e.g., blood. A second excitation light 160B can transmit through some tissues (e.g., skin surface tissues 70, sub-dermal tissues 72) and be reflected 162B by the dye 120 in the catheter tube 112. A third excitation light 160C can transmit through some tissues (e.g., skin surface tissues 70) and be reflected 162C by other tissues (e.g., sub dermal tissues 74). In an embodiment, the first excitation light 160A, second excitation light 160B, and third excitation light 160C can be different wavelengths, or range of wavelengths, from each other. In an embodiment, the first excitation light 160A, second excitation light 160B, and third excitation light 160C can be the same wavelength, or range of wavelengths, which are reflected, transmitted, or absorbed differently based on the structure or fluid that they impinge on. In an embodiment, the second catheter tube 112B can be configured to reflect/fluoresce light differently from the first catheter tube 112A. As such, the system 100 can differentiate between two or more VAD's disposed intravascularly.

Advantageously, the system 100 can facilitate visualizing the placement of the VAD 110, for example placement of the distal tip of the catheter tube 112 within the vessel 74. The system 100 can facilitate visualizing the placement of the catheter tube 112 to ensure correct access to the vessel 74 and mitigate accessing the incorrect vessel, infiltration, extravasation, or backwalling. As used herein, the term “backwalling” includes extending the catheter through a first wall of the vessel 74 to enter the vessel before traversing a back wall of the vessel 74 such that the distal tip is placed outside of the vessel 74.

Advantageously, the system 100 can facilitate visualization of the VAD 110 to prevent placing the distal tip of the catheter tube 112 adjacent a valve 76. As shown in FIG. 2A, since the system 100 can differentiate between blood, vessel tissue 74 and the VAD 110, the system 100 can identify if the distal tip of the catheter tube 112 is disposed adjacent a valve 76. If placed too close to the valve 76, when aspirating blood from the vessel 74, the pressure can cause the valve 76 to close, preventing further blood from being drawn, or can cause damage to the valve 76 itself. Advantageously, as shown in FIG. 2B, the system 100 can identify if a distal tip of the catheter tube 112, e.g., a first catheter tube 112A, is disposed adjacent a bifurcation 78. Aspirating blood from the bifurcation can cause retrograde blood flow through the vessel 74 causing discomfort and complications to the patient.

As shown in FIG. 2C, the dye 120 is included substantially homogenously throughout the material forming the walls of the catheter tube 112. Alternatively, or in addition, the dye 120 is included substantially homogenously throughout a coating disposed on a surface of the catheter tube 112. As the excitation light 160 penetrates the skin surface 70 and surface tissues 72 and impinges on the catheter tube 112, the excitation light 160 reflects/excites the dye 120 causing the dye 120 to emit signal light 162 or reflect reflected light 164. The signal light 162/reflected light 164 contrasts with the surrounding tissues allowing the catheter tube 112 to be imaged.

The optical imaging system 130 using EM radiation provides a high-resolution image of the sub-dermal portions of the VAD 110 against the surrounding tissues and structures, relative to other imaging modalities (e.g., fluoroscopy, ultrasound, etc.). To note, the different compositions of sub-dermal structures will reflect and absorb the excitation light 160 differently allowing these structures, e.g., blood vessels, muscle, dermis, etc. to be imaged relative to the VAD 110 which fluoresces and or reflects differently, as described herein. Advantageously, the system 100 can be used to i) determine correct placement of the sub-dermal structures of the VAD 110, ii) determine the presence/absence and/or degree of thrombus or occlusions on the VAD 110, and iii) determine any physical damage, misalignment, or kinking of the VAD 110.

As shown in FIG. 2D, in an embodiment, when viewed from above the relative concentration of dye 120 is greater towards the lateral sides of the catheter tube 112, i.e., the edges of the catheter tube 112, relative to the central portion of the catheter tube 112. As such, the edges of the catheter tube 112 appear “brighter” relative to the central lumen 118 of the catheter tube 112 since the signal light 162/reflected light 164 is relatively greater at these portions. As such, the structure of the catheter tube 112 can be discerned in the image provided by the optical imaging system 130. Advantageously, the optical imaging system 130 can detect any misplacement or structural abnormalities, such as twisting or kinking of the catheter tube 112 when placed subcutaneously.

For example, incorrect placement of the catheter tube 112 can lead to various problems. Where the distal tip falls short of entering vessel or breaches a far wall of the vessel re-entering the surrounding tissue, fluid can leak into the surrounding tissues causing infiltration or extravasation. Such situations can be avoided if the VAD is imaged beforehand using the system 100. Where the patient returns for repeat treatments, the placement of the VAD 110 can be confirmed prior to starting the treatment to ensure the VAD 110 has not been moved or dislodged during the interim period between placement and treatment. Further, the clinician can confirm the integrity of the VAD device prior to starting the treatment to ensure no damage or kinking has occurred.

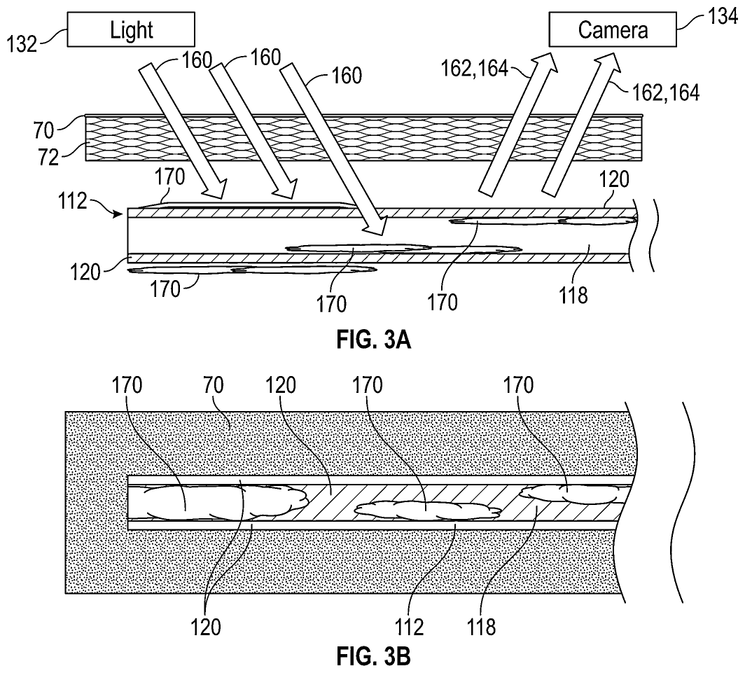

FIG. 3A shows a schematic side view of a portion of the catheter tube 112 disposed sub-dermally and including one or more deposits 170 disposed thereon. FIG. 3B shows a schematic plan view the catheter tube 112 of FIG. 3A when imaged by the optical imaging system 130. The one or more deposits 170 can include biofilms, fibrin sheaths, thromboses, clots or the like. The deposits 170 can form regularly or irregularly over an outer surface of the catheter tube 112, or within the catheter lumen 118, e.g., on an inner surface of the catheter lumen 118.

The deposits 170 affect the reception of excitation light 160 and/or the emission of signal light 162/reflected light 164 from the dye 120 in the VAD 110 and, as shown in FIG. 3B, can affect the “brightness” or contrast of the catheter tube 112 relative to the surrounding tissues, when imaged by the optical imaging system 130. For example, as shown, the deposits 170 appear as contrasting “darker” areas on the image of the catheter tube 112. The effect of the deposits 170 on the dye 120 can provide a distinctive pattern and/or contrast difference which can be used to diagnose the various complications described herein.

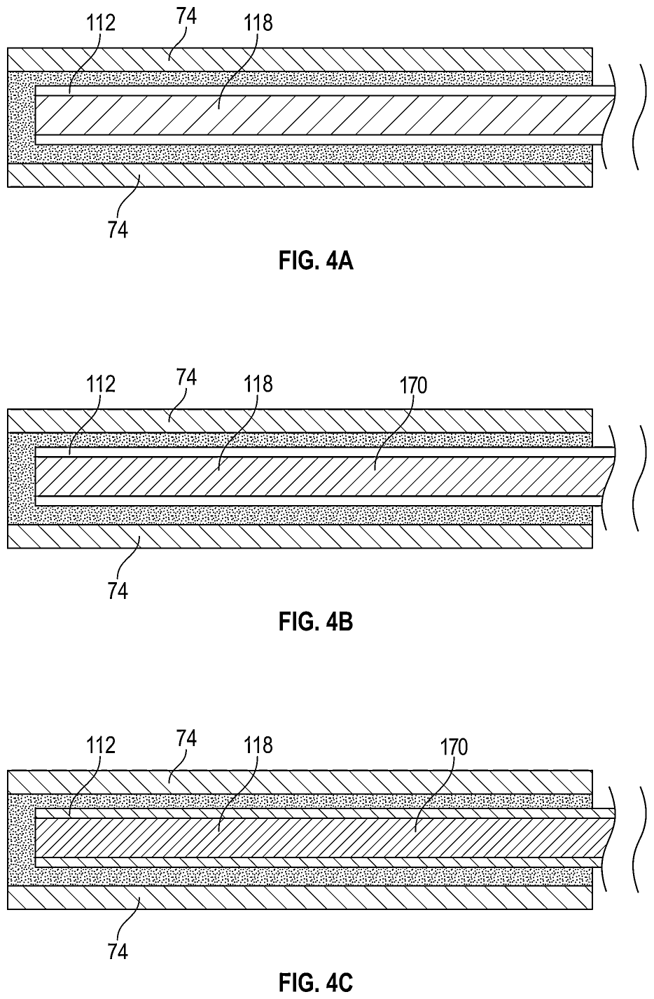

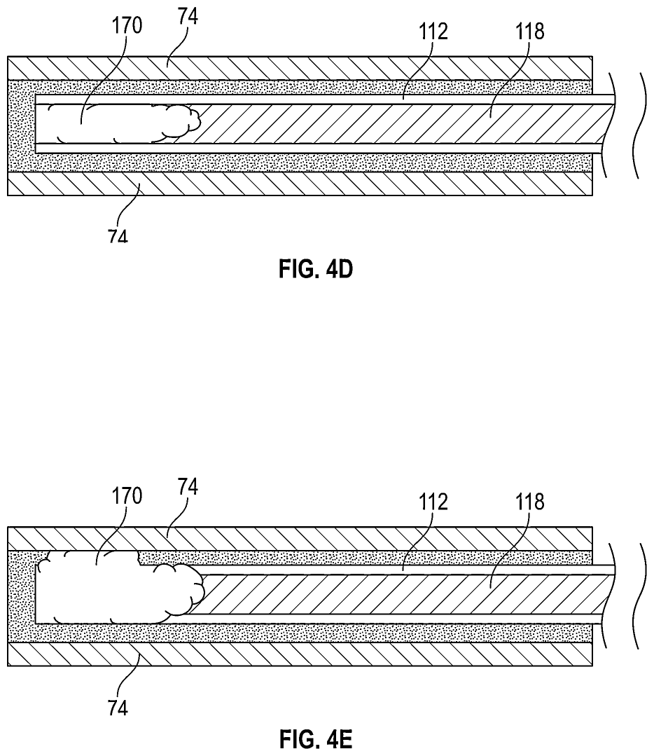

FIGS. 4A-4E show exemplary images of a VAD 110 disposed intravascularly, and the various complications that can be diagnosed based on the differential reflection/emission of EM radiation detected by the system 100. FIG. 4A shows a schematic image of a catheter tube 112 without any deposits 170 or structural deformations. As shown, the vessel walls 74 reflect differently from the blood within the vessel 74. The catheter tube 112 fluoresces/reflects differently again from either of the vessel walls and the blood to differentiate from both. Since the dye 120 is more concentrated towards to the edges of the catheter tube 112 due to the increased depth of material, the walls of the catheter tube 112 can be differentiated from the lumen 118. As such, system 100 can determine the lumen 118 is clear of deposits 170, occlusions, or structural deformations, as described herein. Optionally, the system 100 can use such images as a baseline, or threshold image. The baseline or threshold image can either be established from an image of the VAD 110 soon after placement, from a plurality of images of the VAD 110 over time, or from a predetermined standard of care image stored locally or remotely.

FIG. 4B shows a schematic image of a catheter tube 112 having an intraluminal biofilm deposit 170. Since the intraluminal biofilm obstructs reflected/fluoresced light from the lumen 118 only, and in a relatively uniform manner along the lumen 118, reflected/fluoresced light from the vessel walls 74, blood, and catheter tube wall 112 are relatively unchanged, where the reflected/fluoresced light from the lumen 118 is attenuated relative to the threshold image (FIG. 4A). As such, the system 100 can compare the images of FIGS. 4A, 4B to determine a difference and diagnose an intraluminal biofilm.

FIG. 4C shows a schematic image of a catheter tube 112 having an abluminal biofilm deposit 170. Since the abluminal biofilm obstructs reflected/fluoresced light from the lumen 118 and the catheter tube walls, and in a relatively uniform manner, reflected/fluoresced light from the vessel walls 74 and blood are relatively unchanged, where the reflected/fluoresced light from the lumen 118 and the catheter tube walls 112 are attenuated relative to the threshold image (FIG. 4A). As such, the system 100 can compare the images of FIGS. 4A, 4C to determine a difference and diagnose an abluminal biofilm.

FIG. 4D shows a schematic image of a catheter tube 112 having an intraluminal clot deposit 170. Since the intraluminal clot obstructs reflected/fluoresced light from the lumen 118 only and is concentrated to a specific area of the lumen 118, reflected/fluoresced light from the vessel walls 74, blood, and catheter tube wall 112 are relatively unchanged, where the reflected/fluoresced light from a portion of the lumen 118 is attenuated relative to the threshold image (FIG. 4A). As such, the system 100 can compare the images of FIGS. 4A, 4D to determine a difference and diagnose an intraluminal clot.

FIG. 4E shows a schematic image of a catheter tube 112 having a thrombosis deposit 170. Since the thrombosis deposit 170 is disposed on an outside surface of the catheter tube 112, the deposit 170 obstructs reflected/fluoresced light from the lumen 118 as well as the catheter walls and is concentrated to a specific area of the catheter tube 112. As such, the system 100 can compare the images of FIGS. 4A, 4E to determine a difference and diagnose a fibrin sheath. Reflected/fluoresced light from the vessel walls 74, and blood are relatively unchanged. However, if the thrombosis extends to a wall of the vessel 74 creating a mural thrombosis, or occludes the vessel 74 entirely, reflected light from the vessel walls 74 and blood would also be attenuated in a given area. As such, the system 100 can compare the images of FIGS. 4A, 4D to determine a difference and diagnose a thrombosis or mural thrombosis. These and other complications, structural anomalies, etc. can be diagnosed in a similar manner.

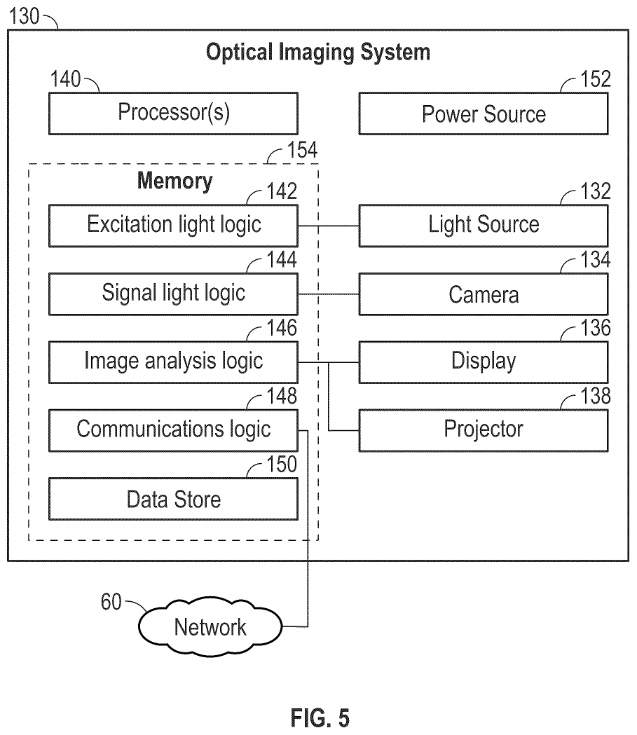

FIG. 5 shows a schematic view of the optical imaging system 130. In an embodiment, the optical imaging system 130 includes one or more processors 140, memory 154, data stores 150, logic modules such as an excitation light logic 142, signal light logic 144, image analysis logic 146, and communications logic 148, as well as hardware such as a light source 132, camera 134, display 136 (e.g., touch screen display or the like), projector 138, a power source 152, or combinations thereof. In an embodiment, the optical imaging system 130 is configured to provide an image based on the signal light 162 and/or reflected light 164 detected by the camera 134. In an embodiment, the optical imaging system 130 can include a handheld device, mobile device, laptop, augmented reality (AR) device, virtual reality (VR) device, or similar device configured to emit excitation light 160 and detect signal light 162/reflected light 164 and provide an image.

In an embodiment, the excitation light logic 142 is communicatively coupled with the light source 132 and configured to control an intensity and/or wavelength of the excitation light 160 provided. In an embodiment, the signal light logic 144 is communicatively coupled with the camera 134 and configured to control features of the camera 134 (e.g., aperture, exposure, etc.) and receive information about the signal light 162 and/or reflected light 164 detected. In an embodiment, the image analysis logic 146 is communicatively coupled with the display 136 and/or projector 138 and configured to receive information from one or more of the excitation light logic 142 and the signal light logic 144 to provide an image of a portion of the VAD 110 and/or surrounding tissues. In an embodiment, the image analysis logic 146 is communicatively coupled with a projector 138 and is configured to project an image onto a skin surface 70 of a patient. For example, to project an image of one or more of the VAD 110, a vessel 74, and surrounding tissues 72, onto the skin surface 70 that is aligned with the one or more of the VAD 110, vessel 74, and surrounding tissues 72 disposed therebelow. The image provided on the display, or projected on to the skin surface can include different colors or contrasts to facilitate visualizing and differentiating the different structures identified.

In an embodiment, the image analysis logic 146 is configured to analyze an image of the VAD 110 and/or compare the current image with previous images stored to the data store 150 to determine changes, for example, changes which may indicate the presence of an occlusion, deposit, structural anomaly, or similar complication described herein. In an embodiment, the differences in the amount of signal light 162/reflected light 164 received can be difficult to discern by subjective assessment of the image displayed. As such, the optical imaging system 130 includes an image analysis logic 146 configured to measure one or more variables of the image (e.g., FIGS. 4B-4E) and compare these one or more variables with corresponding variables extracted from historical or threshold images of the VAD 110 (e.g., FIG. 4A).

In an embodiment, the image analysis logic 146 includes one or more image recognition algorithms, predetermined rulesets and weightings, machine learning schema, artificial intelligence (AI) configured to parse the image received by the camera 134 and compare the information against threshold data to determine a difference. For example, the image analysis logic 146 can parse the image into pixels, assign a numerical value that represents color and intensity for each pixel, and analyzes the relative arrangement for each pixel within the image. In an embodiment, the image analysis logic 146 includes one or more sub-logic modules configured for image feature extraction, and including but not limited to, edge detection, boundary detection, color detection, object identification, combinations thereof, or the like.

The image analysis logic 146 and one or more sub-logic modules can analyze the parsed image to determine different patterns of EM radiation intensities, wavelengths, etc. of the reflected/fluoresced light and their relationships with surrounding pixel data, to determine different boundaries, outlines, shapes within the image and identify different tissues, structures, vessels, fluids, blood, VAD structures (e.g., catheter tube 112, lumen 118, etc.) within the image, and establish a baseline or threshold values for each. A plurality of threshold images can be used to establish differences in image data that are not applicable to complications, which may otherwise register as false positives. Additional images that include complications, such as deposits 170 and/or structural anomalies can indicate deviations in patterns, shape, outline, intensities, and color from the threshold image(s) and can be used to diagnose different complications, as described herein.

The threshold information can be determined from one or more baseline images of the VAD 110, different VAD's from the same or different patients, or standardized image data. In an embodiment, a plurality of images can be taken of the same VAD 110 over a period time. The historical images of the VAD 110 and/or threshold information can be stored in the data store 150, and/or stored remotely and accessed via the communications logic 148 and network 60. In an embodiment, the optical imaging system 130 displays changes in these one or more variables the quantifiable change as a metric on the display 136 of the optical imaging system 130.

In an embodiment, the image analysis logic 146 can include one or more machine learning (ML) schema, AI, or the like to determine threshold information and to determine changes in the image data that might indicate a complication. For example, an ML model can be developed and trained using a plurality of label training data. Labeled training data can include a plurality of images of a VAD that include no deposits 170 or structural abnormalities (FIG. 4A), and include tissue structures, blood, VAD structures (e.g., catheter tube 112, lumen 118 etc.) that are identified and labeled. Further, the labeled training data can include a plurality of images including different deposits 170 having different shapes, outlines, different effects on the reflected/fluoresced light as described herein (e.g., FIGS. 4B-4E). The training data can be stored locally on data store 150 or accessed remotely by communications logic 148 and network 60. Once trained the model can be provided an image from the signal light logic 144 to determine the presence of any deposits 170 and/or structural abnormalities. The system 100 can then provide an image of the VAD 110 and any deposits 170 and/or structural abnormalities identified by the image analysis logic 146 on the display 136/projector 138.

In an embodiment, the system 100 can provide an alert to the user to indicate that a structural anomaly or deposit has been detected. The alert can be a visual, audible, and/or tactile alert. In an embodiment, the system 100 can provide an indication of the type of structural anomaly or deposit detected, e.g., catheter kinking, collapse, biofilm, abluminal film, thrombosis, mural thrombosis, or the like. In an embodiment, the system 100 can further request an input from the user, e.g., visual, audible, or tactile input, to confirm the deposits 170 and/or structural abnormalities identified by the image analysis logic 146 are correct. These inputs can be provided back to the model as training data to further refine the accuracy of the model.

Advantageously, the VAD monitoring system 100 provides an effective means of repeatedly imaging the VAD 110 disposed in situ without exposing the patient or technicians to repeated levels of harmful radiation such as found in fluoroscopic imaging. These images can provide a sharper image relative to ultrasonic imaging and can be used to study the early onset of the formation of deposits on the VAD 110 without disturbing the VAD 110. This can provide valuable insights as to the causes and indicators of the formation of deposits 170 that lead to various VAD complications described herein.

In an embodiment, the image analysis logic 146 of optical imaging system 130 is configured to analyze the image of the VAD 110 measure one or more variables, and determine the presence of, and differentiate between complications described herein, such as infiltration, extravasation, dislodgement, occlusion, loss of patency, infection, catheter kinking, catheter movement, thrombus development, phlebitis, biofilms, fibrin sheaths, intraluminal thromboses, abluminal thromboses disposed on the catheter tube, or the like.

With continued reference to FIG. 1A, in an embodiment, the external portions of the VAD 110, e.g., proximal portions of the catheter tube 112, the catheter hub 114, the dressing 116 can include one or more fiduciary markers 128 configured to facilitate alignment of the optical imaging system 130 in three-dimensional space relative to the catheter tube 112 disposed within the patient.

As will be appreciated, where the differences in the signal light 162/reflected light 164 received are very slight, changes in the position of the light source 132 and the camera 134 of the optical imaging system 130 can affect the results when comparing images with baseline images. For example, where the optical imaging system 130 is positioned further away from the skin surface 70, the excitation light 160 and/or signal light 162/reflected light 164 can show greater attenuation, relative to closer positions. Such attenuation can be misinterpreted as the presence of deposits 170.

As shown in FIG. 1A, in an embodiment, the dressing 116 includes a fiduciary marker 128. Once the VAD 110 has been placed subcutaneously, and the dressing 116 is aligned with the insertion site 90 or external portion of the VAD 110, the fiduciary marker 128 aligns with a portion of the catheter tube 112 disposed therebelow. The light source 132 and/or the camera 134 of the optical imaging system 130 can be aligned with the catheter tube 112 using the fiduciary marker 128 to ensure the optical imaging system 130 is aligned with, and at the same distance from, the catheter tube 112 each time an image is recorded.

In an embodiment, the light source 132 and the camera 134 of the optical imaging system 130 can be included in the same device. In an embodiment, the light source 132 and the camera 134 of the optical imaging system 130 can be provided as separate stand-alone devices. For example, as shown in FIG. 1A, the light source 132 is provided as a first device and the camera 134 and display 136 are provided as a second device, separate from the first device. In an embodiment, the camera 134 and display 136 can be provided as a handheld device such as a smart phone or similar mobile device. In an embodiment, the camera 134 and display 136 can be provided as an AR or VR device or similar wearable device.

Advantageously, a user can manipulate the position and direction of the excitation light 160 emitted from the light source 132 relative to the camera 134 to achieve a clear path for the signal light 162. In an embodiment, the light source 132 can be aligned with a first fiduciary marker 128A and the camera 134 can be aligned with a second fiduciary marker 128B to ensure the relative positioning of the separate light source 132 and the camera 134 are consistent while still allowing a user to modify an angle of attack for the excitation light 160 to provide a clear path for the signal light 162/reflected light 164.

In an embodiment, the light source 132 and the camera 134 are included in a single device and, as such, the relative position and orientation can be predetermined and fixed in place. Advantageously, this can reduce user error in the relative positioning of the light source 132 and camera 134. In an embodiment, one or both of the light source 132 and the camera 134 includes a range finder system, or similar device configured to determine a distance between the light source 132/camera 134 and a skin surface 70. As such, the optical imaging system 130 can indicate to the user at what distance the light source 132/camera 134 are from the skin surface 70, and if the light source 132/camera 134 are at a predetermined, or preferred distance from the skin surface 70.

In an embodiment, the light source 132 is included with a portion of the VAD 110, for example the dressing 116 can include a light source 132 located substantially at the fiduciary marker 128 of FIG. 1A. The light source 132 can be supported against the skin surface, proximate the insertion site and aligned to direct excitation light 160 downwards onto the skin surface 70. For example, as shown in FIG. 1A, the light source 132 can be located at fiduciary marker 128 and provides a ring of illumination within which the camera 134 can be aligned. Advantageously, the light source 132 is maintained in a fixed position relative to the catheter tube 112. In an embodiment, the VAD 110, e.g., dressing 116 can include a power source to provide power to the light source 132 included therewith. In an embodiment, the power source can be included with an external device (e.g., camera 134) and can power the light source 132 by contacting electrodes, induction, or similar suitable means.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A vascular access device (VAD) monitoring system comprising:
a VAD having a catheter tube disposed distally and configured to be disposed subcutaneously, the catheter tube including a dye; and
an optical imaging system including:
a light source configured to emit excitation light; and
a camera configured to detect signal light emitted from the dye when the dye is exposed to the excitation light.

2. The VAD monitoring system according to claim 1, wherein the excitation light includes electromagnetic radiation in a range of 700 nm to 1 mm.

3. The VAD monitoring system according to claim 1, wherein the excitation light includes electromagnetic radiation in a range of 700 nm to 2500 nm.

4. The VAD monitoring system according to claim 1, wherein the dye is formed integrally with a wall of the catheter tube.

5. The VAD monitoring system according to claim 1, wherein the dye is included in a coating disposed on a surface of the catheter tube.

6. The VAD monitoring system according to claim 1, wherein the excitation light has a first wavelength range and the signal light has a second wavelength range, different from the first wavelength range.

7. The VAD monitoring system according to claim 1, wherein the VAD further includes a dressing configured to adhere to a skin surface of a patient and includes a fiduciary marker configured to align one or both of the light source and the camera with a portion of the catheter tube disposed subcutaneously therebelow.

8. The VAD monitoring system according to claim 1, wherein the light source and the camera are provided as a single handheld device.

9. The VAD monitoring system according to claim 1, wherein the light source and the camera are provided as separate stand-alone devices.

10. The VAD monitoring system according to claim 7, wherein the light source is included on the dressing.

11. The VAD monitoring system according to claim 1, wherein the optical imaging system includes an excitation light logic, a signal light logic, and an image analysis logic configured to analyze the signal light detected by the camera and detect a change in the signal light relative to a threshold image.

12. The VAD monitoring system according to claim 11, wherein the threshold image generated from one or more previous images of the VAD is generated by the optical imaging system.

13. The VAD monitoring system according to claim 11, wherein the optical imaging system determines a quantified change in the signal light and displays the quantified change as a metric on a display of the optical imaging system.

14. The VAD monitoring system according to claim 11, wherein the image analysis logic of the optical imaging system is configured to analyze the signal light and generate an image of the catheter tube and determine one or more of an occlusion, a thrombosis, an abluminal biofilm, an intraluminal biofilm, a fibrin sheath, a catheter tube dislodgement, a loss of patency of the catheter tube, a catheter tube collapse, damage to the catheter tube, a proximity of a distal tip of the catheter tube to a vascular valve, or a proximity of the distal tip of the catheter tube to a vascular bifurcation.

15. A method of detecting a complication with a vascular access device (VAD) disposed within a patient, comprising:
providing excitation light to a skin surface of the patient;
impinging the excitation light on a dye included with a catheter tube of the VAD disposed subcutaneously;
emitting signal light from the dye;
detecting the signal light by a camera disposed externally to the patient;
determining an image of the VAD; and
analyzing the image to determine if the complication is present based on a change in the signal light relative to a threshold image data.

16. The method according to claim 15, further including providing the excitation light in an infrared (IR) or near infrared (NIR) spectra.

17. The method according to claim 15, wherein the dye is formed integrally within a wall of the catheter tube.

18. The method according to claim 15, wherein the dye is included in a coating disposed on a surface of the catheter tube.

19. The method according to claim 15, wherein the threshold image data is generated from one or more previous images of the VAD.

20. The method according to claim 15, wherein the complication includes one or more of an occlusion, a thrombosis, an abluminal biofilm, an intraluminal biofilm, a fibrin sheath, a catheter tube dislodgement, a loss of patency of the catheter tube, a catheter tube collapse, damage to the catheter tube, a proximity of a distal tip of the catheter tube to a vascular valve, and a proximity of the distal tip of the catheter tube to a vascular bifurcation.

21. A medical device imaging system, comprising:
a medical device having a catheter tube and including a distal portion disposed intravascularly, the catheter tube including a dye; and
an optical imaging system disposed externally and including a light source, a camera, and a console including one or more logic modules stored within a non-transitory storage medium, the one or more logic modules, when executed by one or more processors, perform operations including:
emitting excitation light from the light source to impinge on the dye included with the distal portion of the catheter tube disposed intravascularly;
detecting signal light emitted from the dye of the distal portion to provide an image;
parsing the image to determine a shape of the distal portion of the medical device; and
analyzing the parsed image against threshold data to determine a presence of a structural abnormality or a deposit.

22. The medical device imaging system according to claim 21, wherein the excitation light includes electromagnetic radiation in an infrared or a near infrared range.

23. The medical device imaging system according to claim 21, wherein the dye is formed integrally with a wall of the catheter tube or is included in a coating disposed on a surface of the catheter tube.

24. The medical device imaging system according to claim 21, wherein the excitation light has a first wavelength range and the signal light has a second wavelength range, different from the first wavelength range.

25. The medical device imaging system according to claim 21, wherein the medical device further includes a dressing configured to adhere to a skin surface of a patient and includes a fiduciary marker configured to align one or both of the light source and the camera with the distal portion of the catheter tube disposed subcutaneously therebelow.

26. The medical device imaging system according to claim 21, wherein analyzing the parsed image against the threshold data further includes training a machine learning model with a plurality of labeled images, the trained machine learning model configured to determine a structural anomaly or a deposit disposed on the medical device and provide an alert to a user.

27. The medical device imaging system according to claim 26, wherein the plurality of labeled images includes labeled images of a medical device including a deposit, labeled images of a medical device including a structural anomaly, and labeled images of a medical device without either of a deposit or a structural anomaly.
